(19) United States
(12) Patent Application Publication
(10) Pub. No.: US 2025/0325252 A1
(43) Pub. Date: Oct. 23, 2025

(54) ULTRASOUND SIGNAL COUPLER

(71) Applicant: Worcester Polytechnic Institute, Worcester, MA (US)

(72) Inventors: Haichong Zhang, Shrewsbury, MA (US); Yichuan Tang, Millbury, MA (US)

(21) Appl. No.: 19/184,355

(22) Filed: Apr. 21, 2025

Related U.S. Application Data
(60) Provisional application No. 63/636,285, filed on Apr. 19, 2024.

(51) Int. Cl. A61B 8/00; G10K 11/28

(52) U.S. Cl. CPC A61B 8/4483; A61B 8/4281; G10K 11/28

(57) ABSTRACT

A reflectional ultrasound device and an acoustic medium for interference and artifact mitigation allows alternative orientation of ultrasound probes for aligning the probe with a sensing surface, a complementary cannula or needle or other position or angle offset from the intended imaging target. A gelatin or similar firm, non-fluid but sound-permeable material holds a shape for passing an ultrasound imaging signal from a transducer or emitter to an acoustic reflector for indirectly focusing onto an imaging target. Cumbersome or unreliable fluid containment is avoided, and the problematic movement of gel substances prone to air infiltration and inconsistent placement are averted. Ultrasound imagers may be oriented for emission parallel to a patient imaging surface, and reflected into internal anatomical structures without substantial interference or signal loss.

### RELATED APPLICATIONS

This patent application claims the benefit under 35 U.S.C. § 119(e) of U.S. Provisional Patent App. No. 63/636,285, filed Apr. 19, 2024, entitled “ULTRASOUND SIGNAL COUPLER,” incorporated herein by reference in entirety.

### BACKGROUND

Ultrasound imaging has been widely used for diagnostics and image-guided intervention. Ultrasound image-guided access is involved in surgical operations such as percutaneous nephrolithotomy (PCNL), percutaneous coronary intervention (PCI), and lumbar puncture (LP). Conventional ultrasound image-guided intervention requires complicated hand-eye collaboration to align the ultrasound image and the needle path.

### SUMMARY

A reflectional ultrasound device and an acoustic medium for interference and artifact mitigation allows alternative orientation of ultrasound probes for aligning the probe with a sensing surface, a complementary cannula or needle or other position or angle offset from the intended imaging target. A gelatin or similar firm, non-fluid but sound-permeable material holds a shape for passing an ultrasound imaging signal from a transducer or emitter to an acoustic reflector for indirectly focusing onto an imaging target. Cumbersome or unreliable fluid containment is avoided, and the problematic movement of gel substances prone to air infiltration and inconsistent placement are averted. Ultrasound imagers may be oriented for emission parallel to a patient imaging surface, and reflected into internal anatomical structures without substantial interference or signal loss.

Configuration herein are based, in part, on the observation than ultrasound imagers enjoy a portability and safety over other imaging mediums, such as MRI Magnetic Resonance Imaging (MRI), Computed Tomography or Computed Axial Tomography (CT/CAT) and X-ray. Ultrasound imaging often employs a liquid or viscous substance between the imaging head, typically an emitter or transducer array, and the imaged region. In medical imaging, this is typically a gel-like substance spread on the epidermal surface where the imaging head glides over. Typically, the ultrasound imaging head (imaging head) emits normal or substantially normal to the imaged region, for rendering images of targets below or obscured. under the skin.

Some imaging approaches complement the ultrasound acoustic signal with an aligned catheter, needle or other instrument. In such contexts, it is beneficial to reflect the ultrasound signal perpendicularly to allow for accompanying instruments. Unfortunately, conventional approaches to ultrasound reflectors suffer from the shortcoming that it is problematic to maintain a gel or liquid between the emitter and the sensing surface because the fluid property tends to flow outside of the path of the ultrasound (US) signal. Further, the gels typically employed tend to retain air bubbles that interfere with US signal propagation. Accordingly, configurations herein provide an acoustic medium with a gelatin or firm texture that holds in place between the imaging head, the reflector and the sensing region in a continuous volume for transmitting the ultrasound signal. In this manner, fluid containments or air bubbles in the sensory path.

In further detail, an acoustic signaling and imaging device as described herein includes an acoustic emitter configured for emitting an ultrasound (US) signal in a direction defined by an orientation of the acoustic emitter, and a reflector aligned with the direction for receiving the US signal and reflecting the US signal towards a sensing surface, where the sensing surface is adjacent an imaging region, typically an external skin surface nearest the imaged region. An acoustic medium between the acoustic emitter and the sensing surface is engaged with the reflector for holding a physical gel-like form for mitigating signal abatement as the US sensing signal and plane passes through the acoustic medium.

### BRIEF DESCRIPTION OF THE DRAWINGS

The foregoing and other features will be apparent from the following description of particular embodiments disclosed herein, as illustrated in the accompanying drawings in which like reference characters refer to the same parts throughout the different views. The drawings are not necessarily to scale, emphasis instead being placed upon illustrating the principles of the invention.

FIG. 1 is a context diagram of an imaging environment suitable for use with configurations herein;

FIG. 2 shows the acoustic medium in the imaging environment of FIG. 1;

FIG. 3 shows imaging of a medical imaging target using a plurality of acoustic reflectors; and

FIG. 4 shows the acoustic medium and device in use in an imaging capture as in FIG. 3.

### DETAILED DESCRIPTION

The configurations disclosed below depict an example ultrasound imaging device and system using a semi-solid acoustic medium for transport of reflected US signals for mitigating artifacting effects of air bubbles and similar interference with fluid and gel mediums. A semi-sold gelatin or firm, gel-like material sufficiently non-fluid to hold a shape avoids containment issues in and around the emitted US signal path.

Ultrasound (US) image-guided access is widely used in surgical operations such as percutaneous nephrolithotomy (PCNL), percutaneous coronary intervention (PCI) and lumbar puncture (LP). One major challenge conventional US image-guided access faces is sustaining proper alignment between the needle path and the US image plane to maximize needle visibility, since the needle and the US transducer are controlled by each individual hand. A conventional reflector-integrated ultrasound (ref-US) image-guided access had been expected to provide by-default alignment of the needle path and the image plane, however issues relating to size, unconventional handling style and encapsulation of an acoustic medium pose difficulties to clinical application of ref-US image-guided access. Configurations herein focus on improving clinical applicability of ref-US image-guided needle access mechanism. A semi-solid gel-like or gelatin medium encapsulates ref-US image-guided access mechanism to reduce imaging artifacts and to prevent medium leakage, while both are common issues in encapsulation using viscous liquid mediums.

In conventional approaches, a liquid medium such as a water-glycerin mixture, along with a bottom layer such as a latex film has been proposed as an encapsulation solution for an imaging facilitation substance. Limitations of this conventional encapsulation include reverberation artifacts, which occur from a difference of acoustic impedances of the liquid medium and the solid layer, floating materials such as air bubbles and contaminating scatters in the liquid medium, and the potential of medium leakage. In a reflector-integrated ultrasound (ref-US) design, configurations herein demonstrate that such a liquid medium is not a necessity and can be replaced by gel-like materials such as gelatin or agarose mixed in appropriate concentrations. Using gel-like materials can avoid the bottom layer, thus eliminating reverberation artifacts. The composition of gel-like materials can be adjusted to mimic the acoustic properties of the actual tissue to optimize the acoustic coupling between the imaging medium and the tissue where an imaging target resides. In addition, floating scatters and medium leakage are also avoided.

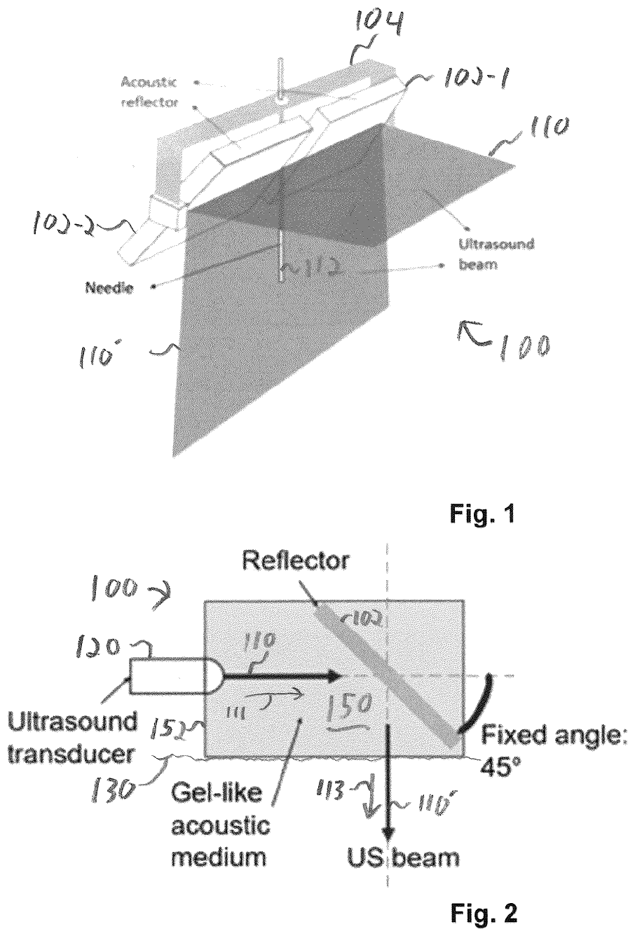

FIG. 1 is a context diagram of an imaging environment 100 suitable for use with configurations herein and shows an acoustic reflector 102-1 . . . 102-2 (102 generally) disposed in an emitted ultrasound (US) signal 110 in a direction defined by an orientation of the acoustic emitter. The acoustic reflector 102 is aligned with the direction for receiving the US signal 110 and reflecting the US signal towards a sensing surface in a reflected signal 110′. In some configurations, a complementary medical tool 112 such as a needle aligns with the imaging plane defined by the reflected signal 110′. The acoustic reflector 102 is supported by a structure 104.

FIG. 2 shows the acoustic medium in the imaging environment of FIG. 1. Referring to FIGS. 1 and 2, the acoustic signaling and imaging device 100 includes an acoustic emitter configured for emitting an ultrasound signal in a direction 111 defined by an orientation of the acoustic emitter 120, which may be an ultrasound transducer, and a reflector 102 aligned with the direction 111 for receiving the US signal and reflecting the US signal towards a sensing surface 130 in a target direction 113, where the sensing surface 130 is adjacent an imaging region. A typical usage would be an epidermal region just above or near a target anatomical feature or organ for imaging. An acoustic medium 150 occupies the transmission space or gap between the acoustic emitter 120 and the sensing surface 130, such that the acoustic medium 150 is engaged with the reflector 102 for mitigating signal abatement.

FIG. 2 depicts the acoustic medium 150 used with ref-US image-guided access attachment with a single, fixed acoustic reflector 102 for exhibiting imaging performance of a gel-like medium such as the acoustic medium 150. Validation trials include filling the containment 152 with various types of gel-like media, including gelatin and agarose, made with different weight ratios of water for evaluating the resulting image quality. Configurations herein depict examples of gelatin and agarose as materials for the gel-like acoustic medium 150. 8%, 12%, and 15% weight ratios were selected for gelatin; and 2%, 3%, and 5% weight ratios were selected for agarose, also using straight water as a baseline.

Validation included computing full-width-at-half-maximum (FWHM) measurements using point targets, along with signal-to-noise ratio (SNR) and contrast-to-noise ratio (CNR) using cyst targets. Equations for SNR and CNR are shown as follows:

$$S N R = 20 {log}_{10} \frac{S i g n a l}{N o i s e} \qquad ( eq . 1 )$$

Here, Signal refers to the maximum signal value in an imaged region, while Noise refers to the standard deviation in the background region:

$$C N R = \frac{\vert {μ}_{targe𝔱} - {μ}_{b a c k} \vert}{\sqrt{{σ}_{target}^{2} + {σ}_{b a c k}^{2}}} \qquad ( eq . 2 )$$

Here, μ_{target} and μ_{back} represent the average pixel values in a small region within the target and in the adjacent background, respectively, while σ_{target} and σ_{back} represent the standard deviation of pixel values in these regions.

Table I shows results of different gel-like media employed as the acoustic medium 150, where:

FWHM stands for Full Width at Half Maximum

SNR stands for Signal-to-Noise Ratio, indicative of the strength of the desired signal (the image information) relative to the background noise or unwanted signals.

CNR stands for Contrast-to-Noise Ratio, a metric that quantifies the ability to distinguish different structures or tissue types within an image based on their contrast relative to the background noise. A higher CNR indicates better image quality and greater ease in detecting subtle differences between regions.

TABLE I

| Medium | FWHM (mm) | SNR (dB) | CNR |
|---|---|---|---|
| Water | 1.35 (0.37) | 34.11 (5.11) | 1.36 (0.21) |
| Gelatin (8%) | 1.46 (0.61) | 35.05 (6.64) | 1.84 (0.39) |
| Gelatin (12%) | 1.10 (0.57) | 32.02 (5.71) | 1.59 (0.28) |
| Agarose (3%) | 1.28 (0.75) | 35.27 (7.15) | 1.77 (0.46) |

*The number out of the parentheses is mean value, the number inside the parentheses is the standard deviation.

In validation of the acoustic medium 150, mechanism, the containment 152 was filled with 3% agarose for validation of the acoustic medium 150 imaging and image guided intervention functionalities. Result of Table I demonstrate that 3% agarose provides the best performance due to its #2 rank in FWHM (1.28 mm), #1 rank in SNR (35.27 dB0, and #2 rank in CNR (1.77).

In other words, the acoustic medium 150 is disposed in simultaneous contact with the acoustic emitter 120, the reflector 102 and the sensing surface and occupies the line of sight between the acoustic emitter 120 and reflector 102, and between the reflector and the sensing surface 130. The acoustic medium 150 may occupy the containment 152 housing the reflector and encapsulating the acoustic medium 150 in an orientation in contact with the acoustic emitter 120 and the sensing surface 130, however since the acoustic medium 150 holds its shape, the housing need not be sealed or fluid resistant, and rather serves to bias the shape of the acoustic medium 150 against and in contact with the acoustic emitter 120, reflector 102 and sensing surface 130. In an example configuration the acoustic medium 150 is formed from one or more of gelatin, agarose, and gel wax. Note that conventional gel often used for US imaging has a liquid property that flows over the sensing surface and cannot hold a vertical form sufficiently long for a transverse mounted acoustic emitter 120. Rather, such conventional gels are intended to form a light contact coating as a conventional US probe glides over a sensed region with the acoustic emitter 120 in a generally normal (90° vertical) orientation.

In many use cases, a transverse mounted acoustic emitter 120 is disposed on the sensing surface 130 and irradiates the US signal in a direction parallel to the sensing surface 130, where the acoustic reflector 102 is oriented at a 45° angle for reflecting the US signal normal to the sensing surface 130. This allows for accompanying medical tools 112 or instruments such as the needle in FIG. 1. However, any suitable angular orientation of the reflector 102 to the signal direction may be performed to accommodate the emitter 120 position.

Configurations of FIGS. 2 and 3 relate to a reflector-integrated attachment for the ultrasound emitter 120 (or transducer), with acoustic reflectors having fixed reflection angles of 45°. To minimize overall size, the disclosed configuration computes the width of the acoustic reflectors and the acoustic path according to the transducer's field of view (FOV), to allow a minimum reflector width to reflect all ultrasound energy. FIG. 3 also demonstrates the use of double reflectors in scenarios where the ultrasound transducer is straight-up and can be handled in the conventional way that a sonographer is familiar with. Dimensions of reflector width and acoustic path are selected based on a distance between the ultrasound transducer and the first and optional second acoustic reflector 102.

FIG. 3 shows imaging of a medical imaging target using the plurality of acoustic reflectors. The path of the acoustic wave is reflected twice and the slot 161 on one of the reflectors 102 allows the needle to pass while keeping aligned with the US image plane. In the configuration of FIG. 3, multiple acoustic reflectors 102-1 . . . 102-2 (102 generally) may be employed to accommodate spacing, angle of approach, and/or pathways for other medical instruments or probes, to name several. In such configurations, a plurality of the acoustic reflectors 102 are disposed in a path of the US signal, such that the US signal 110′ is reflected as intermediate beams or signals 110, 110-N towards an imaging target 140 in an imaged region 142 or anatomic feature based on an aggregate angular orientation of the plurality of acoustic reflectors 102-N.

The acoustic emitter 120 of the US signal 110 may also be defined by any suitable transducer or transducer array based on the imaging target 140. The emitter 120 may therefore include an array of one or more transducer elements, such that each of the transducer elements is configured to send and receive an acoustic signal, wherein a received acoustic signal is indicative of the imaged feature or target 140

A further enhancement is shown by acoustic reflector 102-2, which is reflective of acoustic signals while transparent to optical signals. Optical transparency is complemented by an optical medium projecting optical signals 160 through the acoustic reflector 102-2, via the slot 161 or hole that allow provide an optical view without substantially compromising any reflective capability for acoustic signals. This arrangement allows the ultrasound signal 110 to capture an imaging plane 121 while simultaneously aligned with a visual path and/or needle advancing towards the surgical target 140. This arrangement allows an optical line of sight to align with the needle 160 and slot 161 to be complemented by US signals 110′ from an offset US source.

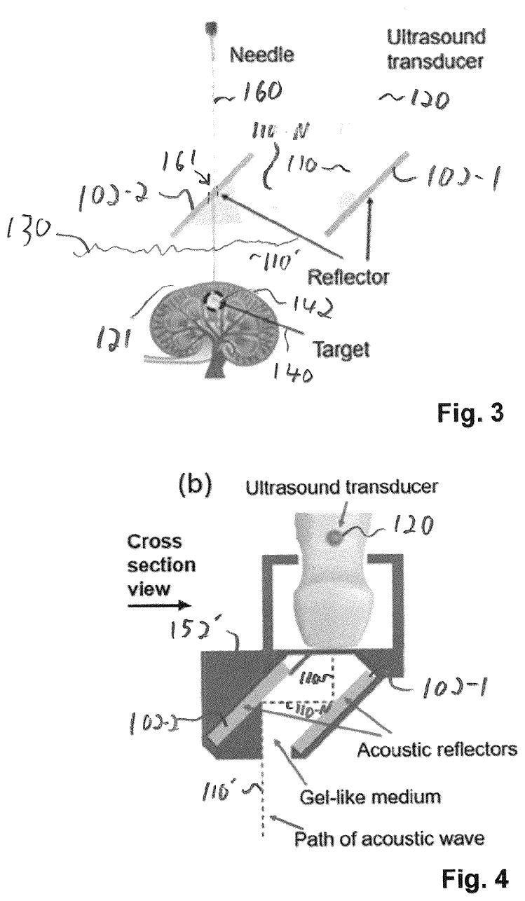

FIG. 4 shows the acoustic medium and device in use in an imaging capture as in FIG. 3. In FIG. 4, a method of gathering an ultrasound (US) image as disclosed above is depicted, including orienting an ultrasound emitter 120 adjacent an imaged feature in an imaging region, where the orientation of the ultrasound emitter 120 or transducer need not be aligned with an emission trajectory of an US signal from the ultrasound emitter. The first acoustic reflector 102 is disposed in a dual reflector containment 152′ in the emission trajectory of the ultrasound emitter is oriented at an angle based on the imaged feature. The containment 152′ has the effect of applying or forming a nonliquid acoustic medium between the ultrasound emitter 120 and the series of acoustic reflectors 102. The angular positioning of the acoustic reflectors 102 has the effect of redirecting the US signal 110 from the ultrasound emitter 120 to the imaged feature or target 140 via aggregate the reflections from the acoustic reflectors 102. It should be noted that the containment 152′ need not be sealed or waterproof, as the acoustic medium 150 has a resilient, non-fluid form that holds a shape under normal gravity, and the containment serves the purpose of positioning the reflectors 102 and for biasing the acoustic medium 150 against the ultrasound emitter 120 for effective transmission of the signal 110 in a continuous reflector path through the acoustic medium to the sensing surface 130.

Variations on mediums which are disposed between ultrasound imaging sources and targets include forms of liquid, gel, gelatin and rigid or solid. Configurations herein present an acoustic medium having a consistency of gelatin for holding a predetermined shape against gravitational or fluid influences that permit medium leakage and flow and a tendency to introduce interference prone air bubbles. The resulting US image-guided access mechanism is therefore optimized for clinical application from two aspects: 1) minimized size and conventional transducer handling enabled by double reflectors; and 2) an acoustic medium defined by a gel-like material which provides effective encapsulation while avoiding reverberation artifacts and medium leakage

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. An acoustic signaling and imaging device, comprising:
an acoustic emitter configured for emitting an ultrasound (US) signal in a direction defined by an orientation of the acoustic emitter;
a reflector aligned with the direction for receiving the US signal and reflecting the US signal towards a sensing surface, the sensing surface adjacent an imaging region; and
an acoustic medium between the acoustic emitter and the sensing surface, the acoustic medium engaged with the reflector for mitigating signal abatement.

2. The device of claim 1 wherein the acoustic medium is disposed in simultaneous contact with the acoustic emitter, the reflector and the sensing surface and occupies the line of sight between the acoustic emitter and reflector, and between the reflector and the sensing surface.

3. The device of claim 1 further comprising a containment housing the reflector and encapsulating the acoustic medium in an orientation in contact with the acoustic emitter and the sensing surface.

4. The device of claim 1 wherein the acoustic medium is selected from the group consisting of gelatin, agarose, and gel wax.

5. The device of claim 3 wherein the acoustic emitter is disposed on the sensing surface and irradiates the US signal in a direction parallel to the sensing surface, the acoustic reflector oriented at a 45° angle for reflecting the US signal normal to the sensing surface.

6. The device of claim 1 further comprising a plurality of acoustic reflectors disposed in a path of the US signal, the US signal reflected towards an imaging target based on an aggregate angular orientation of the plurality of acoustic reflectors.

7. The device of claim 1 wherein the acoustic emitter further comprises an array of one or more transducer elements, each of the transducer elements configured to send and receive an acoustic signal, wherein a received acoustic signal is indicative of an imaged feature.

8. The device of claim 1 wherein the acoustic reflector is transparent to optical signals, further comprising an optical medium projecting optical signals through the acoustic reflector.

9. A method of gathering an ultrasound (US) image, comprising:
orienting an ultrasound emitter adjacent an imaged feature in an imaging region, the orientation unaligned with an emission trajectory of an US signal from the ultrasound emitter;
disposing an acoustic reflector in the emission trajectory of ultrasound emitter, the acoustic reflector oriented at an angle based on the imaged feature;
applying a nonliquid acoustic medium between the ultrasound emitter and the acoustic reflector; and
redirecting the US signal from the ultrasound emitter to the imaged feature via reflection from the acoustic reflector.

10. The method of claim 9 further comprising:
placing the ultrasound emitter on a sensing surface, the sensing surface obscuring the imaged feature;
applying the acoustic medium in communication with the ultrasound emitter, the acoustic reflector and the sensing surface; and
transmitting the US signal from the ultrasound emitter to the acoustic reflector, and from the acoustic reflector to the sensing surface through the acoustic medium.

11. The method of claim 9 wherein the ultrasound emitter is an ultrasound array of one or more ultrasound transducers.

12. The method of claim 9 wherein the acoustic medium is selected from the group consisting of gelatin, agarose, and gel wax.
